(19) United States
(12) Patent Application Publication
(10) Pub. No.: US 2024/0399207 A1
(43) Pub. Date: Dec. 5, 2024

(54) METHOD AND SYSTEM FOR ANALYZING SIGNALS DURING EXERCISE

(71) Applicant: X-trodes LTD, Herzlia (IL)

(72) Inventors: Yael HANEIN, Caesarea (IL); David RAND, Haifa (IL); Stas STEINBERG, Katzrin (IL); Lilah INZELBERG YIFA, Ein Vered (IL); Ziv PEREMEN, Tel Aviv (IL)

(73) Assignee: X-trodes LTD, Herzlia (IL)

(21) Appl. No.: 18/690,734

(22) PCT Filed: Aug. 30, 2022
(86) PCT No.: PCT/IL2022/050948

§ 371 Date: Mar. 10, 2024

Related U.S. Application Data
(60) Provisional application No. 63/242,051, filed on Sep. 9, 2021.

(51) Int. Cl. A63B 24/00

(52) U.S. Cl. CPC A63B 24/0062; A63B 2024/0071; A63B 2220/836; A63B 2225/50

(57) ABSTRACT

A system for analyzing electrophysiological signals comprises a plurality of electrodes which are adherable to a skin of a subject, and which comprises a ground electrode and a set of signal electrodes. The system also comprises a processor in communication with the electrodes. The processor has a circuit configured to receive from the signal electrodes a first set of signal channels when the ground electrode is disconnected and a second set of signal channels when the ground electrode is active, to compare powers above baseline among the first and the second set of signal channels, and to determine an attachment state of each electrode based on the comparison.

### RELATED APPLICATION

This application claims the benefit of priority of U.S. Provisional Patent Application No. 63/242,051 filed on Sep. 9, 2021, the contents of which are incorporated herein by reference in their entirety.

### FIELD AND BACKGROUND OF THE INVENTION

The present invention, in some embodiments thereof, relates to an electrophysiology and, more particularly, but not exclusively, to a method and system for analyzing electro-physiological signals, and optionally and preferably their fidelity, obtained from an individual during physical exercise or rehabilitation session.

Skin electrodes are a common noninvasive tool used to record chemical and electrophysiological signals from the surface of the body. Electrical activity applications include electroencephalography (EEG), electromyography (EMG), electrocardiogramhy (ECG) and Electrooculography (EOG). Surface EMG (sEMG) in particular has been suggested for a very wide range of applications such as brain-machine interfacings and facial sEMG to record emotions. International application publication No. WO2017/090050 discloses an array of dry electrodes, which can be used for measuring EMG, EEG, or ECG signals from the skin by placing the array on the skin such that there is a direct contact between the electrodes and the surface. The disclosed electrodes are attached to one side of a double sided adhesive film, and the opposite side of the film is attached to the skin. The electrodes are exposed to the skin via openings formed on the film.

### SUMMARY OF THE INVENTION

According to an aspect of some embodiments of the present invention there is provided a system for analyzing electrophysiological signals. The system comprises: a plurality of electrodes which are adherable to a skin of a subject, and which comprises a ground electrode and a set of signal electrodes. The system also comprises a processor in communication with the electrodes. The processor has a circuit configured to receive from the signal electrodes a first set of signal channels when the ground electrode is disconnected and a second set of signal channels when the ground electrode is active, to compare powers above baseline among the first and the second set of signal channels, and to determine an attachment state of each electrode based on the comparison.

According to some embodiments of the invention the processor is configured to receive data pertaining to noise signals when the ground electrode and each of the signal electrodes are inactive, and to determine a baseline power based on the noise signals.

According to some embodiments of the invention the processor is configured to generate output pertaining to the attachment state on a display.

According to some embodiments of the invention the processor in configured to divide the set of signal electrodes into two distinct subsets based on the attachment state, to determine muscle activity based on signal channels corresponding exclusively to one of the subsets, and to generate output pertaining to the muscle activity on the display.

According to some embodiments of the invention the processor is configured to receive locations of the electrodes, and to identify activation patterns of active muscles based on the received locations and the determined muscle activity. According to some embodiments of the invention the processor is configured to include in the output a displayable map of the locations and the activation patterns.

According to some embodiments of the invention the processor is configured to generate a warning if a parameter characteristic to the muscle activity is outside a predetermined range of thresholds.

According to some embodiments of the invention the system comprises an inertia measurement device configured to generate signals pertaining to motion characteristics of the electrodes, wherein the processor is in communication with the inertia measurement device, and is configured to analyze synchronization between signals received from the electrodes and signals received from the inertia measurement device, and to determine muscle activity based on the analysis.

According to some embodiments of the invention the inertia measurement device is configured to generate the signals over a plurality of signal channels, each channel corresponding to one motion characteristic.

According to some embodiments of the invention the processor is configured to receive input pertaining to an organ to which the electrodes are attached, and to select signal channels of the inertia measurement device based on the input.

According to some embodiments of the invention the system comprises an external inertia measurement device configured to generate signals pertaining to motion characteristics of an exercise apparatus, wherein the processor is in communication with the external inertia measurement device, and is configured to analyze synchronization between signals received from the electrodes and signals received from the inertia measurement device, and to determine muscle activity based on the analysis.

According to an aspect of some embodiments of the present invention there is provided a system for analyzing electrophysiological signals. The system comprises: a plurality of electrodes adherable to a skin of a subject, and an inertia measurement device configured to generate signals pertaining to motion characteristics of the electrodes. The system also comprises a processor in communication with the electrodes and the inertia measurement device. The processor has a circuit configured to analyze synchronization between signals received from the electrodes and signals received from the inertia measurement device, to generate output pertaining to muscle activity based on the analysis.

According to some embodiments of the invention the system comprises acquisition circuitry for receiving the signals from the electrodes and wirelessly transmitting data pertaining to the signal to the processor.

According to some embodiments of the invention the processor is configured to receive input pertaining to an organ to which the electrodes are attached, and to select signal channels of the inertia measurement device based on the input.

According to an aspect of some embodiments of the present invention there is provided a method of analyzing electrophysiological signals during exercise. The method comprises: adhering to a skin of a subject a plurality of electrodes which comprises a ground electrode and a set of signal electrodes, establishing electrical communication between a processor and the signal electrodes but not the ground electrode, receiving from the signal electrodes a first set of signal channels, establishing electrical communication between the processor and the ground electrode in addition to the electrical communication between the processor and the signal electrodes, and receiving from the signal electrodes a second set of signal channels. The method also comprises comparing, by the processor, powers above baseline among the first and the second set of signal channels, and determining an attachment state of each electrode based on the comparison.

According to some embodiments of the invention the method comprises digitizing the signals and wirelessly transmitting data pertaining to the signal to the processor.

According to some embodiments of the invention the digitization and the transmission is by acquisition circuitry, wherein the method comprises receiving from the acquisition circuitry noise signals when the ground electrode and each of the signal electrodes are inactive, and determining a base line power based on the noise signals.

According to some embodiments of the invention the method comprises dividing the set of signal electrodes into two distinct subsets based on the attachment state, determining muscle activity based on signal channels corresponding exclusively to one of the subsets, and displaying the muscle activity on a display.

According to some embodiments of the invention the method comprises receiving locations of the electrodes, identifying activation patterns of active muscles based on the received locations and the determined muscle activity, and displaying a map of the locations and the activation patterns on the display.

According to some embodiments of the invention the method comprises generating a warning if a parameter characteristic to the muscle activity is outside a predetermined range of thresholds.

According to some embodiments of the invention the parameter, based on which the warning is issued, comprises at least one of level of pain, force exerted by a muscle, level of muscle fatigue, and asymmetry of muscle activity.

According to some embodiments of the invention the method comprises receiving from an inertia measurement device signals pertaining to motion characteristics of the electrodes, analyzing synchronization between signals received from the electrodes and the signals received from the inertia measurement device, and determining muscle activity based on the analysis.

According to some embodiments of the invention the method comprises receiving from an external inertia measurement device signals pertaining to motion characteristics of an exercise apparatus, analyzing synchronization between signals received from the electrodes and signals received from the external inertia measurement device, and determining muscle activity based on the analysis.

According to an aspect of some embodiments of the present invention there is provided a method of analyzing electrophysiological signals. The method comprises: adhering to a skin of a subject a plurality of electrodes adherable, and an inertia measurement device, receiving from the electrodes electrophysiological signals, receiving from the inertia measurement device signals pertaining to motion characteristics of the electrodes, analyzing synchronization between signals received from the electrodes and signals received from the inertia measurement device, and generating output pertaining to muscle activity based on the analysis.

According to some embodiments of the invention the inertia measurement device generates the signals over a plurality of signal channels, each channel corresponding to one motion characteristic, and the method comprises receiving input pertaining to an organ to which the electrodes are attached, and selecting signal channels of the inertia measurement device based on the input.

According to some embodiments of the invention the electrodes are adhered to a body portion selected from a group consisting of a portion of a neck, a portion of an arm, a portion of a leg, a portion of a hand, a portion of a foot, a portion of a torso, and a portion of a head.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a system for analyzing electrophysiological signals, according to some embodiments of the present invention;

FIG. 2 is a schematic illustration of an arrangement of electrodes and acquisition circuitry, according to some embodiments of the present invention; and

FIGS. 3A-E show signals collected during in experiments performed according to some embodiments of the present invention by electrodes attached to a skin of an individual.

### DESCRIPTION OF SPECIFIC EMBODIMENTS OF THE INVENTION

The present invention, in some embodiments thereof, relates to electrophysiology and, more particularly, but not exclusively, to a method and system for analyzing signals obtained from an individual during physical exercise.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present embodiments comprise electrodes that are attached to the skin of a subject and collect electrophysiological signals pertaining to muscle activity in a region below the skin. The present embodiments can also comprise a processor having a circuit configured to execute program instructions that receive the signals from the electrodes and analyze them. The present embodiments are particularly useful for analyzing muscle activity of an individual during indoor or outdoor exercise or physical rehabilitation. In these embodiments, the circuit optionally and preferably generates output data based on the analysis and transmits the output data, for example, to a mobile device, to allow the individual to view his or her muscle activity during the exercise.

In some embodiments of the present invention, the circuit determines, based on the analysis, the attachment state of one or more of the electrodes. In some embodiments of the present invention, the circuit receives from an inertia measurement device signals pertaining to motion characteristics of the electrodes and/or of an exercise apparatus, and analyzes synchronization between signals received from the electrodes and signals received from the inertia measurement device, to determine muscle activity based on the analysis. In some embodiments the circuit receives time stamps from external devices, such as cameras, optical sensors, accelerometers, dynamometers, etc., for the purpose of synchronization with the electrophysiological signal.

The electrodes of the present embodiments measure electrical activity in different parts of a region of the skin under examination. From patterns of electrical activity, the circuit can determine locations of active muscles or active segments of muscles. The circuit can optionally and preferably also determine a condition of the muscle or a group of muscles, e.g., whether it is non-functional, ill-functioning, or improperly functioning, and provide the individual with information pertaining to the condition of the muscle or group of muscles, e.g., on a display of a mobile device.

The circuit can also be used to induce functionality in non-functioning muscles or improve functionality in ill-functioning or improperly-functioning muscles or muscle groups if used in a bio-feedback configuration.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processor circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

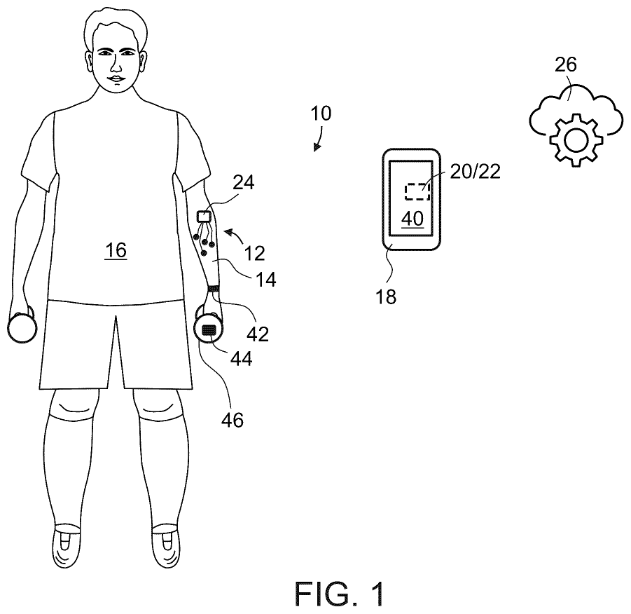

FIG. 1 is a schematic illustration of a system 10 for analyzing electrophysiological signals, according to some embodiments of the present invention. System 10 comprises a plurality of electrodes 12 adherable to a skin 14 of a subject 16, and a processor 20, in communication with dedicated acquisition circuitry 24 connected to electrodes 12. A circuit 22 is configured to perform the signal analysis and computation operations described herein. In the schematic illustration of FIG. 1, which is not to be considered as limiting, processor 20 is not physically connected to electrodes 12. In these embodiments, dedicated acquisition circuitry 24 has a communication functionality that receives the signals from electrodes 12 and transmits them wirelessly to processor 20. The acquisition circuitry 24 optionally and preferably digitizes the signals, before the transmission, and transmits digital data pertaining to the signals to processor 20.

Processor 20 can, in some embodiments of the present invention, be embodied as a CPU of a mobile device 18. The mobile device 18 can be any of a variety of computing devices, such as, but not limited to, cell phone, smartphone, handheld computer, laptop computer, notebook computer, tablet device, notebook, or the like. In some embodiments of the invention the mobile device is a smart phone, and some embodiments of the invention the mobile device is a smart watch. Processor 20 can alternatively be embodied as a desktop computer. The mobile device or desktop computer can also communicate with a server processor in a cloud computing facility 26 in which case at least some of the computation and processing operations described herein are executed by the server processor and the mobile device or desktop computer is used by the computer server to display information to the user.

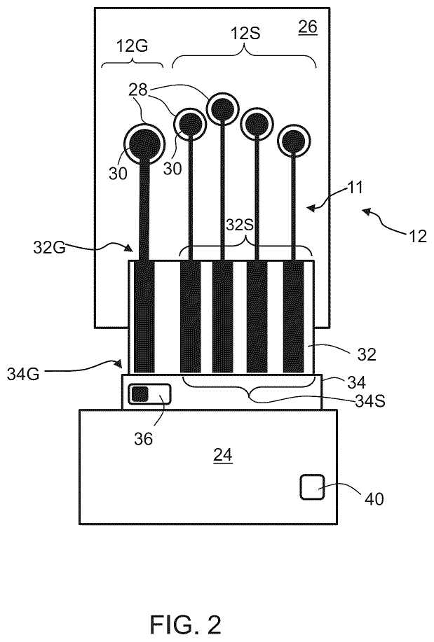

Electrodes 12 and acquisition circuitry 24 are better shown in FIG. 2. Each of electrodes 12 include a conductive line 11 and a sensing portion 30. Although FIG. 2 illustrates each of conductive lines 11 as a straight line, this need not necessarily be the case, since the conductive lines of electrodes 12 can have any shape. Electrodes 12 are optionally and preferably printed electrodes, such as, but not limited to, printed carbon electrodes. Other conductive printed electrodes are also contemplated. The electrodes are optionally and preferably deposited, more preferably printed, on a substrate 26 characterized by a Young's modulus of less than 30 MPa, e.g., from about 1 MPa to about 30 MPa. A representative example of a material suitable for use as a substrate is, without limitation a polyurethane. The diameter of the electrodes comprises is typically from about 3 mm to about 10 mm. The inventors found that such dimensions allow high density, while maintaining low noise levels and good conformity with the skin. The thickness of the substrate is typically from about 60 to about 150 μm, e.g., 80 μm.

As used herein the term “about” refers to ±10%

Substrate 26 can comprise, for example, a double-sided adhesive film bonded to an additional film. The electrodes 12 are preferably on one side of substrate 26, and the opposite side of the substrate 26 is attachable to the skin 14 (not shown, see FIG. 1). In these embodiments, the double-sided adhesive film comprises a plurality of openings 28 to expose the sensing portion 30 of each electrode 12 to the skin 14 contacting the opposite side of substrate 26. Preferably, the conductive lines 11 are isolated from the skin 14 by substrate 26. Electrodes 12 typically comprise a set of signal electrodes generally shown at 12S, and one or more ground electrodes shown at 12G. Ground electrode(s) 12G is/are preferably integral, namely they are formed on the same substrate 26 as electrodes 12S.

The coupling between electrodes 12 and acquisition circuitry 24 is preferably by a male-female pair 32, 34 of multichannel electrical connectors. In some embodiments of the present invention the rigid part of the multichannel connector is multi-layered such that 34S and 34G are intermixed. Each of connectors 32 and 34 has a first part 32S, 34S, for establishing connection of the signal electrodes 12S to circuitry 24, and ensuring that the signal sensed by each individual electrode is channeled to circuitry 24 as a separate signal channel, and a second part 32G, 34G, for establishing connection between ground electrode 12G and circuitry 24.

The second part 32G and 34G is illustrated at the end of pair 32, 34, but it can alternatively be located between two adjacent conductive lines 11 of set 12S.

Connectors 32 and 34 are preferably detachable from each other. Preferably, but not necessarily, connector 32 is not detachable from electrodes 12, and connector 34 is not detachable from acquisition circuitry 24.

In some embodiments of the present invention at least one of connectors 32 and 34 comprises a switch 36 for controlling the electrical communication between second part 32G of connector 32 and second part 34G of connector 34. Switch 36 can be a mechanical switch or any component that is capable of changing a state of electrical communication. For example, switch 36 can be a magnetic switch, or an electronic logic operated by acquisition circuitry 24, or a zero-ohm jumper component that can be either soldered or otherwise connected to connector 34.

Switch 36 is typically a two-state switch having an ON state at which there is an electrical communication between parts 32G and 34G (and therefore between ground electrode 12G and circuitry 34), and an OFF state at which parts 32G and 34G (and therefore ground electrode 12G and circuitry 34) are electrically isolated from each other. Switch 36 may be controlled by acquisition circuitry 24 thus allowing a totally automated testing session prior to the onset of the measurement.

Switch 36 thus provide system 10 with the ability to provide a separate control over the electrical communication of circuitry 24 with ground electrode 12G and signal electrodes 12S. Specifically, when connectors 32 and 34 are disconnected, there is no electrical communication between circuitry 24 and any of the electrodes 12; when connectors 32 and 34 are connected and switch 36 is brought to its OFF state, there is electrical communication between circuitry 24 and electrodes 12S, but not with electrode 12G; and when connectors 32 and 34 are connected and switch 36 is brought to its ON state, there is electrical communication between circuitry 24 and electrodes 12S, as well as electrode 12G.

The advantage of having a control over the electrical communication between the ground electrode 12G and circuitry 24, separately from the control over the electrical communication between the signal electrodes 12S and circuitry 24, is that it allows determining the signal fidelity prior to a data collection session, as will be explained in greater detail below.

Connector 32 is preferably monolithic, but embodiments in which first parts 32S and 32G are provided as two separate connectors, are also contemplated. Similarly, connector 34 is preferably monolithic, but embodiments in which second parts 34S and 34G are provided as two separate connectors, are also contemplated. When first parts 32S and 32G are provided as two separate connectors, and also when second parts 34S and 34G are provided as two separate connectors, it is not necessary for connector 32 or 34 to include switch 36, since the communications between second parts 32G and 34G can be controlled by connecting and disconnecting them from each other. Yet, embodiments in which switch 36 is included in part 32G or 34G also when those parts are provided as separate connectors, are also contemplated.

Acquisition circuitry 24 comprises a communication circuit 40, providing wireless communication. The wireless communication can be according to any known protocol, such as, but not limited to, Bluetooth, Z-wave, ZigBee, ANT, WIFI, GPRS, GSM, CDMA, 3G, 4G, and 5G.

In a typical use of system 10, electrodes 12 and acquisition circuitry 24 are provided separately from each other, so that connector 34 (at the side of acquisition circuitry 24) is not connected to connector 32 (at the side of electrodes 12). Individual 16 (FIG. 1) attaches substrate 26 to the skin 14, and also mounts acquisition circuitry 24, e.g., by means of a belt or adhesive, at sufficient proximity from substrate 26 to allow the aforementioned pairs of connectors to be connected to each other. Instructions for attaching the electrodes 12 and circuitry 24 can be provided to the individual 16, for example, by means of an app installed on the mobile device 18. Typically, such instructions are accompanied by cartoons or images or animated cartoons or a video stream.

In some embodiments of the present invention processor 20 receives from signal electrodes 12S, optionally and preferably by acquisition circuitry 24, a first set of signal channels when ground electrode 12G is disconnected. Processor 20 also receives from signal electrodes 12S a second set of signal channels when ground electrode 12G is connected. This can be achieved by instructing the individual, for example, using the app installed on the mobile device 18, to first establish a connection between connectors 34 and 32 while keeping parts 32G and 34G electrically isolated from each other (e.g., by setting switch 36 at its OFF state, or by not connecting parts 32G and 34G to each other, when they are provided as separate connectors), allow acquisition circuitry 24 to transmit data to processor 20 for a predetermined period of time (e.g., 20-60 second), then establish an electrical communication between connectors 32G and 34G (e.g., by bringing switch 36 to its ON state, or by physically connecting parts 32G and 34G to each other, when they are provided as separate connectors), and allow acquisition circuitry 24 to transmit data to processor 20 for an additional predetermined period of time (e.g., 20-60 second). The app may also issue a que to the individual when to establish the electrical communication, e.g., when a sufficient amount of data has been transmitted to processor 20.

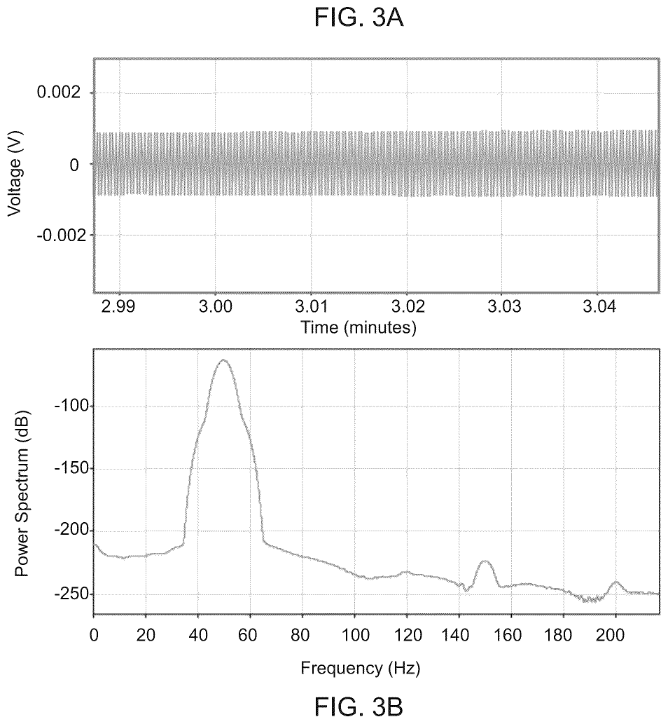

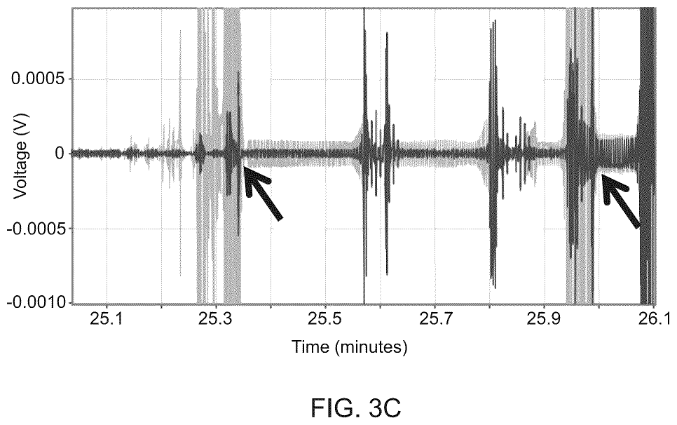

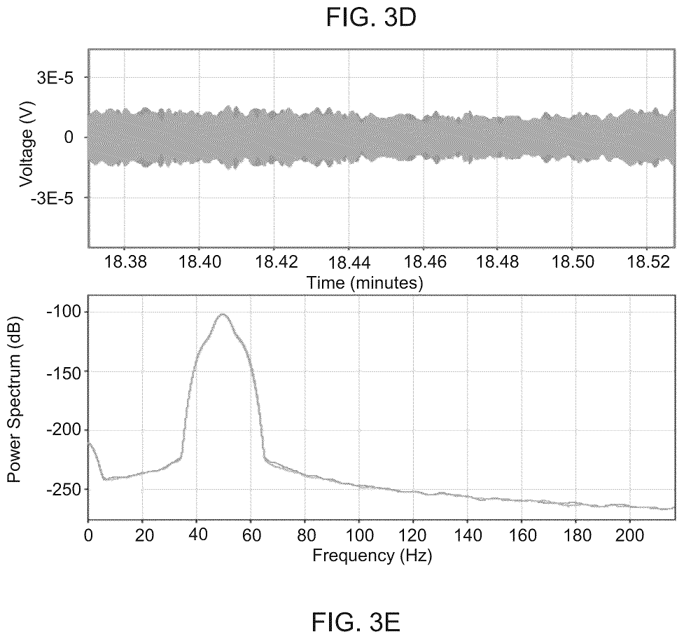

The Inventors found that by comparing the powers above baseline among the first and the second set of signal channels, the attachment state of each of electrodes 12, particularly each of the signal electrodes 12S, can be determined. This will now be explained with reference to FIGS. 3A-C.

FIGS. 3A and 3B show a single recorded channel, which is typical to the first set signal channels as received from the signal electrodes 12S, in the time domain (FIG. 3A) and in the frequency domain (FIG. 3B). Since the first set of signal channels is acquired while the ground electrode 12G is not connected, the signal channels exhibit a peak at a noise frequency that depends on the fundamental electrical grid frequency (for example, about 50 Hz in, e.g., Europe, about 60 Hz in, e.g., the U.S.). In the representative example shown in FIGS. 3A and 3B, the peak is at a noise frequency of 50 Hz. Since the noise signal is due to the fundamental electrical grid frequency, it is highly correlated among the signal channels (which are not shown in FIGS. 3A and 3B, but are generally of similar shapes in the time and frequency domains).

In the experiment shown in FIGS. 3A-C, all the electrodes were firstly properly connected to the skin. Thereafter, one signal electrode was deliberately disconnected from the skin, to demonstrate the effect of such disconnection on the noise level.

FIG. 3C shows a single recorded channel of the second set of signal channels, as received from the signal electrodes 12S, in the time domain. Shown is the signal channel that was deliberately disconnected during the experiment. The two thick arrows on FIG. 3C indicate the time points at which the electrode was disconnected.

Since the second set of signal channels is received while the ground electrode 12G is connected, the signal channels at the onset of the measurement exhibit a reduction of power at the noise frequency, albeit still above the baseline. By comparing the powers above baseline among the first and the second set of signal channels, the attachment state of the individual electrodes is detected. For example, at the time points at which the signal electrode was disconnected (the two thick arrows on FIG. 3C) the signal received over the channel corresponding to this electrode exhibited an abrupt increase. The increase is due to the noise level that dominates the signal level. In some embodiments of the present invention the channel the channel can also be subjected to spectral analysis to ensure that the increase is due to a peak at the noise frequency (50 Hz, in the present example). The channels that are dominated by noise, also become uncorrelated with the other electrodes, and such lack of correlation indicates that the respective signal electrodes are attached improperly.

Thus, the present embodiments exploit the noise generated by the wide area synchronous grid in order to determine the connection state of the signal electrodes. In particular, the present embodiments successfully determine the connection state without the need to measure the impedance for each channel. This is advantageous over conventional electrophysiological systems that can only determine the connection state by employing a special impedance measuring circuit, which makes the system bulky and cumbersome.

Once the attachment states of the electrodes are detected, the processor 20 optionally generates output pertaining to the attachment states on a display 40. Display 40 can be the display of the mobile device 18, as illustrated in FIG. 1, or a separate display, for example, a display of a desktop computer.

The comparison of the powers above baseline among the first and the second set of signal channels can also be used to determine the attachment state of the ground electrode 12G. For example, when the connection of the ground electrode 12G does not result in a sufficient reduction of the power above the baseline at the noise frequency, the system can determine that the ground electrode 12G is not properly attached, or that connector 34 is not properly connected.

Typically, the system determines that the ground electrode 12G and that connector 34 is properly connected, when the ratio between the powers of the second and first sets of signals at the noise frequency is less than a predetermined ratio threshold. When the ratio is not less than the predetermined ratio threshold, the system issues an alert indicating that ground electrode 12G malfunctions. The individual 16 can then better secure the ground electrode 12G and/or ensure that connector 34 is properly connected. The predetermined ratio threshold is optionally and preferably selected based on the length of the electrodes. A typical value for the predetermined ratio threshold is about 0.6 or about 0.5 or about 0.4. In some embodiments of the present invention the ratio threshold adaptive.

The baseline of signal channels provided by acquisition circuitry 24 can be recorded on a memory medium accessible by processor 20. Alternatively, the baseline can be determined by processor 20. In these embodiments, processor 20 receives signals from acquisition circuitry 24 before a connection is established between connectors 34 and 32. Thus, for example, the app can provide the individual 16 with an instruction to initially (or at any other desired point in time) leave both parts of connectors 34 and 32 unconnected for a predetermined period of time (e.g., 20-60 second). During this period, there is no communication between any of electrodes and acquisition circuitry 24, and so at this stage circuitry 24 transmits pure noise channels. The power of the noise signals is significantly lower when all electrodes are disconnected from acquisition circuitry 24, than when electrodes 12S are connected and electrode 12G is disconnected, and is also lower than the power at the noise frequency when all the electrodes are connected. The power of the noise signals when all electrodes are disconnected is therefore determined by processor 20 as the baseline power.

FIGS. 3D and 3E show noise signals as received from acquisition circuitry 24 when all electrodes are disconnected, in the time domain (FIG. 3D) and in the frequency domain (FIG. 3E), demonstrating a significantly lower power at the noise frequency compared to FIGS. 3A-B. Thus, the power shown in FIGS. 3D and 3E can be determined by processor 20 as the baseline power, and be used in the comparison between the first and second signal channels.

Once the attachment states of the signal electrodes 12S are determined, processor 20 optionally and preferably divide them into two distinct subsets, based on their attachment states. This can be done by a thresholding procedure based on the correlation in terms of the power at the noise frequency. Specifically, a correlation coefficient can be calculated for each electrode relative to one or more electrodes (e.g., all other electrodes), and compared to a predetermined correlation coefficient threshold. Processor 20 then classifies each signal electrode into one subset when the calculated correlation coefficient is above the threshold, and into another subset otherwise. In cases in which the calculated correlation coefficient of none of the signal electrode is above the threshold processor 20 preferably issues an alert, for example, by means of display 40.

In various exemplary embodiments of the invention processor 20 processes the signals from the first subset of signal electrodes (the subset that contains those electrodes for which the calculated correlation coefficient is above the threshold) so as to determine muscle activity. Preferably, processor 20 determine the muscle activity exclusively based on the signals from the first subset of signal electrodes. In other words, processor 20 does not use the signals from the second subset of signal electrodes (the subset that contains those electrodes for which the calculated correlation coefficient is not above the threshold) for determining the muscle activity.

Electrical activity of a muscle can be found by employing a technique that can sense a change in bioelectrical potential which can be picked-up from the surface of the skin. Examples include, but are not limited to, electroencephalography (EEG), electrocardiogra Electrooculography (EOG) (recording eye movement), electro-olfactography (EOLG), and electromyography (EMG). In some preferred embodiments, at least one of EMG, and ECG is used. Preferably, muscle activity is determined by executing a blind source separation procedure to the digital data pertaining to the signals from the electrodes (preferably only the first subset of the signal electrodes).

Representative examples of blind source separation procedures suitable for the present embodiments including, without limitation, independent component analysis (ICA), fast independent component analysis (fICA), principal component analysis, singular value decomposition, dependent component analysis, non-negative matrix factorization, low-complexity coding and decoding, stationary subspace analysis, common spatial pattern analysis and any combination thereof.

In some embodiments of the present invention independent component analysis is employed. Independent component analysis is a technique that separates the source signals from a mixed signal without any information about how the source signals are mixed, on the assumption that the source signals are statistically independent of each other.

In some embodiments of the present invention processor 20 obtains locations of the electrodes and uses these locations in the analysis. The locations of the electrodes can be obtained based on the shape and size of the electrodes and the location on the skin 14 to which substrate 26 is attached. For example, processor 20 can uses an app installed on the mobile device 18 for generating an instruction regarding the location on the skin 14 to which substrate 26 is to be attached and also regarding the orientation in which the substrate 26 is to be attached at this location, or, more preferably, generates a set of controls on the display 40 that allow the individual 16 to select from a list of predetermined locations on the skin 14 to which substrate 26 is to be attached, and provide the individual withy instructions regarding the orientation in which the substrate 26 is to be attached at the selected location. The list of predetermined locations can comprise, for example, one or more elements of a group consisting of a portion of a face, a portion of a neck, a portion of an arm, a portion of a leg, a portion of a hand, a portion of a foot, a portion of a torso, and a portion of a head.

Also contemplated, are embodiments in which processor 20 determine the locations of the electrodes by means of image processing. For example, following the attachment of substrate 26 to the skin, the individual can use the mobile device 18 to image substrate 26 and an identifiable skin portion nearby substrate 26. Processor 20 can receive the captured image and apply image processing to identify the skin portion and the electrodes and/or substrate 26, and determine the locations of the electrodes relative to the skin portion.

The locations of the electrodes are typically used by processor 20 to identify activation patterns of active muscles based on the determined muscle activity. For example, processor 20 can generate a map of muscle activation patterns and locations of active muscles or segments of active muscles. Such a map can be displayed on display 40. Processor 20 can additionally display an image of a body portion and/or a graphical representation of the electrodes, and the map of muscle activation patterns can overlay such an image and/or graphical representation of the electrodes.

In some embodiments of the present invention processor 20 generates a warning if a parameter characteristic to the determined muscle activity is outside a predetermined range of thresholds. Representative examples of such parameters including, without limitation, level of pain, force exerted by a muscle, level of muscle fatigue, and asymmetry of muscle activity. Referring again to FIG. 1, system 10 optionally and preferably comprises an inertia measurement device 42, configured to generate signals pertaining to motion characteristics of electrodes 12. Inertia measurement device 42 can be provided as a separate device, as illustrated in FIG. 1, or be mounted on or integrated with acquisition circuitry 24. Inertia measurement device 42 can alternatively be a component in mobile device 18, in which case mobile device 18 is mounted on individual 16 at the vicinity of electrodes 16, for example, by means of a belt or a wearable device holder.

Inertia measurement device 42 can be of any type known in the art. Inertia measurement device 42 can include embedded instrumentation such as one or more gyroscopes, angular accelerometers, velocity meters, or other inertial sensors. For example, inertia measurement device 42 can comprise a spinning wheel gyroscope, such as, but not limited to, a dynamically tuned gyroscope, a rate gyroscopes, or a rate integrating gyroscope. In some embodiments of the present invention inertia measurement device 42 is a micro electro mechanical system (MEMS)-based measurement device.

Typically, inertia measurement device 42 detects motion characteristics, such as, but not limited to, an acceleration, a rate of change of acceleration, a rate of change in attitude (e.g., pitch, roll, and/or yaw rates). Inertia measurement device 42 can contain several (e.g., three) accelerometers and several (e.g., three) gyroscopes. The accelerometers are placed within the enclosure of device 42 such that their measuring axes are orthogonal to each other, allowing them to measure an acceleration vector (e.g., a three-dimensional acceleration vector). The gyroscopes can also be placed within the same enclosure such that their measuring axes are orthogonal to each other, allowing them to measure rotation rates.

Inertia measurement device 42 transmits signals pertaining to the motion characteristics to processor 20. In some embodiments of the present invention inertia measurement device 42 generates the signals over a plurality of signal channels, each channel corresponding to one motion characteristic. For example, device 42 generates separate signal channels to different components of the acceleration, and separate signal channels to changes in different attitudes. In some embodiments of the present invention, inertia measurement device 42 generates the signals over six signal channels, three channels corresponding to acceleration along three orthogonal linear axes, and three channels corresponding to changes in attitude along three orthogonal angular axes.

Processor 20 analyzes synchronization between signals received from the electrodes 12, and signals received from the inertia measurement device 42, and determines the muscle activity based on this analysis. For example, processor 20 can give more weight to signals from electrodes 12 that are synchronized with the signals from device 42 than for other signals. When device 42 provides the signals over a plurality of channels, processor 20 can receive input pertaining to the organ to which the electrodes are attached, and select signal channels of device 42 based on this input. For example, when substrate 26 is attached to the thigh of the individual, electrodes 12 can sense muscle activity of the rectus femoris muscle, and processor 20 can select for the analysis those channels of device 42 that are indicative of the knee joint angle or hip angle.

The input can be provided by the individual, e.g., using an app installed on mobile device 18. For example, in cases in which the app provides the individual with a set of controls that allow the individual 16 to select from a list a location to which substrate 26 is to be attached, the selected location can be used by processor 20 as the organ to which the electrodes are attached.

System 10 may, alternatively or additionally, comprise an external inertia measurement device 44, that generates signals pertaining to motion characteristics of an exercise apparatus 46. Apparatus 46 is simplified in FIG. 1 as a dumbbell, but may be embodied as any exercise apparatus known in the art, such as, but not limited to, a treadmill, a bicycle machine, an oval exerciser, a stationary bicycle, a stair climbing machine, a cross-country skiing analog machine, a jack, a rowing machine, and the like. External inertia measurement device 44 can be of any of the types described above with respect to device 42.

Processor 20 can receive signals from device 44 and analyze synchronization between the signals received from electrodes 12 and the signals received from device 44, and determine the muscle activity based on this analysis. For example, similarly to the procedure described above with respect to device 42 (mounted on the body of individual 16) processor 20 can give more weight to signals from electrodes 12 that are synchronized with the signals from external device 44 than for other signals.

In some embodiments of the present invention processor 20 uses the signals from the inertia measurement device(s) without correlating the signals from the electrodes to a video stream. This is advantageous over conventional EMG systems in which in order to accurately determine muscle activity, the signals sensed from the skin are correlated to a video stream that captures the motion of the individual.

In some embodiments of the present invention processor 20 provides the individual with instructions to mount the inertia measurement device(s), based on the type of exercise the individual is performing. For example, processor 20 can uses an app installed on the mobile device 18 to generates a set of controls on the display 40 that allow the individual 16 to select from a list of predetermined exercise activities the type of exercise that the individual is about to perform. The processor can receive the selection from the controls, and issue, e.g., on display 40, an instruction which inertia measurement device to use, and where to mount it. As representative examples, when the exercise is a vertical jump, the processor can issue an instruction to mount the inertia measurement device on or near the knee of the individual, and when the exercise is cycling on a bicycle machine, the processor can issue an instruction to mount the inertia measurement device on the crank of the bicycle machine.

The analysis performed by processor 20 optionally and preferably involves feeding digital data to a trained machine learning procedure, and receiving from the machine learning procedure output pertaining to the muscle activity. The digital data that are fed to the machine learning procedure represent any the signals described herein, including, without limitation, the signals received from signal electrodes 12S, the signals received from the inertia measurement device(s), when employed. The digital data may also represent one or more of the aforementioned inputs, such as the organ to which the electrodes are attached, and the locations of the electrodes on the organ.

The trained machine learning procedure can be combined with other data analysis procedure, such as, but not limited to, a blind source separation procedure as further detailed hereinabove. For example, the output of the blind source separation procedure can be used as input to the machine learning procedure.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors. The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features. The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features. The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact. Information gain is one of the machine learning methods suitable for feature evaluation. The information gain is based on a quantity known as entropy, which is a measure of impurity in a collection of training instances. The reduction in entropy of the target feature that occurs by knowing the values of a certain feature is called information gain. Information gain may be used as a parameter to determine the effectiveness of a feature in determining the muscle activity.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on the muscle activity, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the muscle activity. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Neural networks are a class of algorithms based on a concept of inter-connected “neurons.” In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some embodiments of the present invention is a trained machine learning procedure. A machine learning procedure can be trained according to some embodiments of the present invention by feeding a machine learning training program with the digital data that of a cohort of individuals for which the muscle activity has been determined using a laboratory equipment. Once the data are fed, the machine learning training program generates a trained machine learning procedure of a selected type which can then be used without the need to re-train it.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for analyzing electrophysiological signals, comprising:
a plurality of electrodes adherable to a skin of a subject, and comprising a ground electrode and a set of signal electrodes; and
a processor, in communication with said electrodes and having a circuit configured to receive from said signal electrodes a first set of signal channels when said ground electrode is disconnected and a second set of signal channels when said ground electrode is active, to compare powers above baseline among said first and said second set of signal channels, and to determine an attachment state of each electrode based on said comparison.

2. The system according to claim 1, comprising an acquisition circuit for receiving said signals from said electrodes and wirelessly transmitting data pertaining to said signal to said processor.

3. The system according to claim 2, wherein said processor is configured to receive from acquisition circuit data pertaining to noise signals when said ground electrode and each of said signal electrodes are inactive, and to determine a baseline power based on said noise signals.

4. The system according to claim 1, wherein said processor is configured to generate output pertaining to said attachment state on a display.

5. (canceled)

6. The system according to claim 4, wherein said processor in configured to divide said set of signal electrodes into two distinct subsets based on said attachment state, to determine muscle activity based on signal channels corresponding exclusively to one of said subsets, and to generate output pertaining to said muscle activity on said display.

7. (canceled)

8. The system according to claim 6, wherein said processor is configured to receive locations of said electrodes, to identify activation patterns of active muscles based on said received locations and said determined muscle activity, and to include in said output a displayable map of said locations and said activation patterns.

9. (canceled)

10. The system according to claim 6, wherein said processor is configured to generate a warning if a parameter characteristic to said muscle activity is outside a predetermined range of thresholds.

11. (canceled)

12. The system of claim 10, wherein said parameter comprises at least one of level of pain, force exerted by a muscle, level of muscle fatigue, synchronization and asymmetry of muscle activity.

13. (canceled)

14. The system according to claim 1, comprising an inertia measurement device configured to generate signals pertaining to motion characteristics of said electrodes, wherein said processor is in communication with said inertia measurement device, and is configured to analyze synchronization between signals received from said electrodes and signals received from said inertia measurement device, and to determine muscle activity based on said analysis.

15. (canceled)

16. The system according to claim 14, wherein said inertia measurement device is configured to generates said signals over a plurality of signal channels, each channel corresponding to one motion characteristic.

17. (canceled)

18. The system according to claim 16, wherein said processor is configured to receive input pertaining to an organ to which said electrodes are attached, and to select signal channels of said inertia measurement device based on said input.

19. (canceled)

20. The system according to claim 1, comprising an external inertia measurement device configured to generate signals pertaining to motion characteristics of an exercise apparatus, wherein said processor is in communication with said external inertia measurement device, and is configured to analyze synchronization between signals received from said electrodes and signals received from said inertia measurement device, and to determine muscle activity based on said analysis.

21. (canceled)

22. A system for analyzing electrophysiological signals, comprising:
a plurality of electrodes adherable to a skin of a subject;
an inertia measurement device, configured to generate signals pertaining to motion characteristics of said electrodes;
a processor, being in communication with said electrodes and said inertia measurement device, and having a circuit configured to analyze synchronization between signals received from said electrodes and signals received from said inertia measurement device, to generate output pertaining to muscle activity based on said analysis.

23. The system according to claim 22, comprising acquisition circuitry for receiving said signals from said electrodes and wirelessly transmitting data pertaining to said signal to said processor.

24. The system according to claim 22, wherein said processor is configured to receive input pertaining to an organ to which said electrodes are attached, and to select signal channels of said inertia measurement device based on said input.

25. A method of for analyzing electrophysiological signals during exercise, comprising:
adhering to a skin of a subject a plurality of electrodes comprising a ground electrode and a set of signal electrodes;
establishing electrical communication between a processor and said signal electrodes, but not said ground electrode, to receive from said signal electrodes a first set of signal channels;
establishing electrical communication between said processor and said ground electrode, in addition to said electrical communication between said processor and said signal electrodes, to receive from said signal electrodes a second set of signal channels; and
by said processor, comparing powers above baseline among said first and said second set of signal channels, and determining an attachment state of each electrode based on said comparison.

26. The method according to claim 25, comprising digitizing said signals and wirelessly transmitting data pertaining to said signal to said processor.

27. (canceled)

28. The method according to claim 25, comprising dividing said set of signal electrodes into two distinct subsets based on said attachment state, determining muscle activity based on signal channels corresponding exclusively to one of said subsets, and displaying said muscle activity on a display.

29-35. (canceled)

36. The method according to claim 25, comprising receiving from an inertia measurement device signals pertaining to motion characteristics of said electrodes, analyzing synchronization between signals received from said electrodes and said signals received from said inertia measurement device, and determining muscle activity based on said analysis.

37-39. (canceled)

40. The method according to claim 25, comprising receiving from an external inertia measurement device signals pertaining to motion characteristics of an exercise apparatus, analyzing synchronization between signals received from said electrodes and signals received from said external inertia measurement device, and determining muscle activity based on said analysis.

41-45. (canceled)
